(19) United States
(12) Patent Application Publication
(10) Pub. No.: US 2019/0320946 A1
(43) Pub. Date: Oct. 24, 2019

(54) COMPUTER-IMPLEMENTED DYNAMICALLY-ADJUSTABLE AUDIOMETER

(71) Applicants: Matthew Bromwich, Gloucester (CA); Julian Bromwich, Ottawa (CA); Heikki Koivikko, Ottawa (CA); Erica Sampson, Richmond (CA)

(72) Inventors: Matthew Bromwich, Gloucester (CA); Julian Bromwich, Ottawa (CA); Heikki Koivikko, Ottawa (CA); Erica Sampson, Richmond (CA)

(21) Appl. No.: 16/386,421

(22) Filed: Apr. 17, 2019

(30) Foreign Application Priority Data
Apr. 18, 2018 (CA) 3002004

(51) Int. Cl. A61B 5/12; A61B 5/00; H04R 3/04

(52) U.S. Cl. CPC A61B 5/123; H04R 3/04; A61B 5/7475

(57) ABSTRACT

A computer-implemented method for dynamically adjusting the operation of an automated audiometer during a hearing test of a patient comprising the steps of: (i) collecting or loading pre-collected personalization settings of the patient before the hearing test; (ii) adjusting parameters of the hearing test to accord with the personalization settings of the patient; (iii) commencing the hearing test; (iv) actively monitoring and analyzing at least one input factor during the hearing test; (v) adjusting operation of the hearing test if the at least one input factor meets a pre-defined triggering scenario; (vi) repeating steps (iv) to (v) until the hearing test has been completed by the patient or stopped by the audiometer; and (vii) analyzing results of the hearing test.

### FIELD OF THE INVENTION

The present invention relates to a portable computer-implemented audiometer. More particularly, the present invention relates to a portable computer-implemented automated audiometer that is capable of dynamically adjusting its operation based on environmental monitoring, personalization settings, interaction analysis, and data analysis.

### BACKGROUND OF THE INVENTION

Most traditional audiometers are designed to operate only within the confines of a sound-insulated booth under strict environmental parameters; and function (most commonly) under the manual control of an audiologist, or much less frequently in a fully automated fashion (or some combination of the two).

Thus, if a person desired to create and utilize a portable, computer-implemented audiometer that is capable of operating outside a sound-insulated booth, one can appreciate that it would be very important to monitor how a person under testing responds to outside factors that could negatively affect the quality and accuracy of the test (such as external noise pollution). As such, when operating an audiometer outside the confines of a sound-insulated booth, it is important to be able to dynamically adjust the operation of an audiometer to compensate for outside factors, even while using high attenuation headphones, and especially when the audiometer is to operate in a fully-automated fashion.

The present invention relates to the implementation of a portable, computer-implemented audiometer that is capable of overcoming problems associated with operating an audiometer without the presence of a skilled audiologist who would otherwise monitor the patient's understanding of the test and adjust the protocol accordingly, as well as overcoming problems associated with operating an audiometer outside of a sound-insulated booth that would otherwise enforce strict environmental parameters. More particularly, the present invention relates to the implementation of a portable, computer-implemented audiometer having dynamic adjustment capabilities, and is therefore hereinafter referred to as a “reactive” audiometer.

### SUMMARY OF THE INVENTION

The present invention provides for the implementation of a portable, fully automated, computer-implemented, reactive audiometer that is capable of dynamically adjusting its operation during testing based on environmental monitoring, personalization settings, interaction analysis, and data analysis.

The present invention provides a computer-implemented method for dynamically adjusting an audiometer's operation based on input factors from 4 categories comprising: (i) actively monitoring selected environmental factors before and/or during the test; (ii) freshly collecting or reloading pre-collected personalization preferences before the test; (iii) actively monitoring and analyzing the user's interactions with the audiometer during the test; (iv) scoring the reliability of the collected data; and (v) adjusting the operation of the audiometer based on analysis on one or more input factors.

In one embodiment, the present invention provides a computer-implemented method for dynamically adjusting the operation of an automated audiometer during a hearing test of a patient comprising the steps of: (i) collecting or loading pre-collected personalization settings of the patient before the hearing test; (ii) adjusting parameters of the hearing test to accord with the personalization settings of the patient; (iii) commencing the hearing test; (iv) actively monitoring and analyzing at least one input factor during the hearing test; (v) adjusting operation of the hearing test if the at least one input factor meets a pre-defined triggering scenario; (vi) repeating steps (iv) to (v) until the hearing test has been completed by the patient or stopped by the audiometer; and (vii) analyzing results of the hearing test.

The personalization settings of the patient may include any one or more of demographics, previously existing medical conditions, including tinnitus, colour-blindness, mentation level, physical dexterity, and existing hearing loss, prior noise exposure, usage of hearing-aids or assistive devices, general technology experience level, personal test protocol preferences, spoken or written languages, literacy level, cultural background, job description, and prior hearing test performance.

The step of adjusting parameters of the hearing test to accord with the personalization settings of the patient may include any one or more of adjusting a colour-palette when the patient is colour-blind, selecting pulsed or warble tone when the patient has tinnitus, enabling an interaction technique that requires less precision when the patient has physical dexterity limitations, selecting age-appropriate graphical themes and encouragements for the patient, and selecting testing algorithm modes, including self-paced or semi-assisted modes, to accommodate age or mental agility needs of the patient.

The input factor may include one or more of an environmental factor and/or interactions of the patient with the audiometer.

The environmental factor may include any one or more of ambient background noise (decibel-level), timing and constancy characteristics of background noise, distracting sound, temperature, humidity, lighting levels, and non-auditory vibrations. The interactions of the patient with the audiometer may comprise any one or more of patterned answers, malingering, inconsistent responses, misunderstanding of a task, attention level, and pace and style of responses.

The steps of actively monitoring and analyzing one or more environmental factors and adjusting operation of the hearing test may comprise the steps of: (i) using hardware-integrated or external microphones to ascertain and record ambient noise levels at selected frequencies over selected time intervals; (ii) comparing the recorded ambient noise levels against sound levels and durations that are permitted to occur during the hearing test; (iii) determining if the recorded noise levels should prevent the hearing test from proceeding to a next step, or if a current step should be invalidated; and (iv) based on the determination at step (iii), either pausing the hearing test until ambient noise levels improve to an acceptable level, or immediately re-testing an invalidated step, or marking the current step for subsequent retesting and immediately moving on to the next step of testing.

The steps of actively monitoring and analyzing one or more interactions of the patient with the audiometer and adjusting operation of the hearing test may comprise the steps of: (i) monitoring patient interactions with the audiometer during the hearing test to detect any one or more of response patterns, malingering, inconsistent responses, misunderstanding of the task, attention drift, and cognition issues; (ii) determining if the patient interactions meets the pre-defined triggering scenario for the one or more of response patterns, malingering, inconsistent responses, misunderstanding of the task, attention drift, and cognition issues; and (iii) if the pre-defined triggering scenario for the one or more of response patterns, malingering, inconsistent responses, misunderstanding of the task, attention drift, and cognition issues is met at step (ii), modifying operation of the audiometer in accordance with pre-defined responses to the triggering of the one or more of response patterns, malingering, inconsistent responses, misunderstanding of the task, attention drift, and cognition issues.

The step of modifying operation of the audiometer in accordance with pre-defined responses may include presenting challenges to the patient designed to confirm or refute presence of one or more of response patterns, malingering, inconsistent responses, misunderstanding of the task, attention drift, and cognition issues.

The operation of the hearing test may be further modified to increase a number of hearing trials required to acquire a threshold to compensate for malingering or inconsistent responses, when malingering or inconsistent responses has been confirmed.

The step of modifying operation of the audiometer in accordance with pre-defined responses may include any one of pausing the test and prompting for an attendant to intervene, presenting instructional guidance to the patient, or presenting practice tests if misunderstanding of the task or attention drift is detected.

In another embodiment, the present invention provides a computer program product comprising a computer readable memory storing computer executable instructions thereon that when executed by a computer performs the above-noted method.

In yet another embodiment, the present invention provides a portable computer that performs the above-noted computer-implemented method.

In yet a further embodiment, the present invention provides a computer-based audiometer that performs the above-noted computer-implemented method.

In another embodiment, the present invention provides a computer-implemented method for dynamically adjusting the operation of an automated audiometer during a hearing test of a patient comprising the steps of: (i) defining input factor producers from one or more categories of Environmental Monitoring, Patient information, Interaction Analysis and Data Analysis, whereby each such input factor producer is capable of continuously monitoring a defined set of sensor or data inputs for a pre-defined triggering scenario during operation. of the hearing test; (ii) defining a reaction that may occur during operation of the hearing test upon triggering of the pre-defined triggering scenario for each such input factor producer; (iii) encoding the pre-defined triggering scenario and corresponding reaction for each input factor producer into a ruleset that defines which reaction or sequence of reactions should occur for each pre-defined triggering scenario; (iv) commencing the hearing test and running the input factor producers; (v) c: ntinuously evaluating the input factor producers, wherebyif a pre-defined triggering scenario for an input factor producer is met, an input factor token for thetriggered input factor producer placed into an factor container; (vi.)

continuously evaluating the ruleset based on contents of the input factor container to determine which reaction or reactions should be executed, executing those pre-defined. reactions indicated by the ruleset, and clearing the input factor container; and repeating steps (v) to (vi) until the hearing test is ended.

### BRIEF DESCRIPTION OF THE DRAWINGS

FIG. 1 displays a high-level general system overview showing some of the capabilities of the reactive audiometer in accordance with one embodiment of the present invention.

FIG. 2 displays a general architectural overview of a reactive audiometer in accordance with one embodiment of the present invention.

FIG. 3 displays a component diagram of the input factors, reactions, and helper components that are used in the pseudocode provided in FIGS. 5a to 5i to implement one embodiment of the present invention.

FIG. 4A displays a chart listing the various Input Factors and Reaction categories, providing convenient shorter names for the purposes of illustration herein.

FIG. 4B displays an overview of an example Reactive Rule Set that may be implemented in accordance with one embodiment of the present invention.

FIGS. 5a to 5i display pseudocode for implementing the core components of a reactive audiometer in accordance with one embodiment of the present invention.

FIG. 5a displays pseudocode that defines a CriticalSection class, PredicateReactionPair class, Context class, and InputFactorProducer abstract class, in accordance with one embodiment of the present invention.

FIGS. 5b to 5e each display pseudocode showing an implementation example of an input factor producer for each of the 4 categories of input factors, in accordance with one embodiment of the present invention.

FIG. 5f displays pseudocode relating to the audiometer's main loop that executes a series of queued protocols, in accordance with one embodiment of the present invention.

FIG. 5g displays pseudocode for showing how an Algorithm abstract class can be defined, in accordance with one embodiment of the present invention. Here the Algorithm abstract class is defined as an extension of the Protocol class.

FIG. 5h displays pseudocode showing an example of how a HughsonWestlake algorithm may define its performReaction function in accordance with one embodiment of the present invention.

FIG. 5i displays pseudocode showing a possible implementation for defining the rules shown in FIG. 4b into an array call reactionRuleSet in accordance with one embodiment of the present invention.

### DETAILED DESCRIPTION OF THE INVENTION

The following description is presented to enable a person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the scope of the invention as claimed. Thus, the present invention is not intended to be limited to the embodiments disclosed, but is to be accorded the widest scope consistent with the principles and features disclosed herein.

The following definitions will assist with an understanding of the nature of the invention as described.

Air testing—an air conduction test where sound is delivered through the air to the test subject's ear canal.

Ambient background noise levels—(i.e. room noise level) These are the background sound pressure levels at a given location.

Asymmetric hearing loss—A hearing loss where there is considerable hearing threshold difference between ears, typically 15 dB or more.

Audiologist—A health care professional who is trained to evaluate hearing loss and related disorders.

Audiologist-assisted protocol—A hearing testing mode where an audiologist (or a skilled person) may be used to complete an automated hearing test. The assisting person in this case inputs the responses based on test subject's answers but does not make algorithm-impacting decisions on behalf of the reactive audiometer.

Audiometer—A machine used for evaluating hearing loss.

Bone testing—Bone testing is similar to an air conduction test except the sound is delivered via bone oscillator which produces a physical vibration. A bone oscillator is placed on the test subject's skull or mastoid and the vibration produces an auditory sensation for the test subject.

CAPD—Central Auditory Processing Disorder (CAPD), also known as Auditory Processing Disorder (APD), is an umbrella term for a variety of disorders that affect the way the brain processes auditory information.

Central Masking Effect—A situation where a hearing threshold is elevated due to a masking effect caused by another sound signal (whether it is due to external noise or a masking sound produced by the audiometer).

CircularBuffer—in computer science, is a data structure that uses a single, fixed-size buffer as if it were connected end-to-end.

Demographic Norms—The range of data expected to be observed in a particular sector of a population.

Discrim—(i.e. Speech Discrimination Test) A measure of the ability to recognize a spoken word if it is uttered loud enough for the hearer to detect it as a sound.

DTT—(i.e. Digit Triplet Test) A particular speech intelligibility test that tests the patient's ability to hear spoken numbers above a background noise.

Fast Fourier Transform (FFT)—is an algorithm that samples a signal over a period of time (or space) and divides it into its frequency components.

Hearing Threshold—The sound level below which a person's ear is unable to detect any sound at the tested frequency.

Malingering—(i.e. cheating) The fabrication of hearing loss.

Masking—Auditory masking occurs when the perception of one sound is affected by the presence of another sound.

Modified Hughson Westlake Algorithm—(i.e. Hughson Westlake) A widely used technique for acquiring a patient's hearing threshold across a range of frequencies by presenting tones in a prescribed sequence of volume levels.

MPANL—Maximum Permissible Ambient Noise Level. Various standards like ANSI, ASHA, OSHA specify maximum ambient sound levels (measured in dB SPL) that are permissible (at particular test frequencies) during a hearing test.

Pure-tone—An acoustic pressure wave with amplitude and frequency defined by a sine wave.

Pure tone audiometry (PTA)—The primary hearing test used to identify hearing threshold levels.

Psychoacoustic tests—Hearing tests targeting the psychological and physiological aspects of the sense of hearing in an unimpaired ear.

Self-paced protocol—A hearing test mode where the test subject is controlling the pace of the test by controlling the sound presentation themselves. The frequency and hearing level are controlled by the reactive audiometer while the test subject is responsible for presenting the sound by using the audiometer's controls and responding whether they heard the sound or not.

Sound-insulated Booth—A small room used for hearing tests that is soundproofed to keep out external sounds.

Speech-in-noise—The general classification for any speech intelligibility test that presents speech while playing a background noise.

SRT—(i.e. Speech Reception Threshold) The intensity at which speech is recognized as meaningful symbols; in speech audiometry, it is the decibel level at which 50% of spondee words (metrical foot consisting of two long (or stressed) syllables) can be repeated correctly by the subject.

Stenger test—A test for detecting simulation of unilateral hearing impairment, in which a tone below the admitted threshold is presented to the test ear and a tone of lesser intensity is presented to the other ear.

Threshold-seeking Algorithm—Any algorithm with the intention of determining Hearing Thresholds. Common algorithms are Hughson Westlake and Bekesy.

Tinnitus—A ringing or buzzing in the ears.

The reactive audiometer of the present invention is capable of dynamically adjusting an audiometer's operation in a manner that approximates or even improves upon adjustments that would be made by a human audiologist if/when performing hearing testing in an environment outside of a sound-insulated booth.

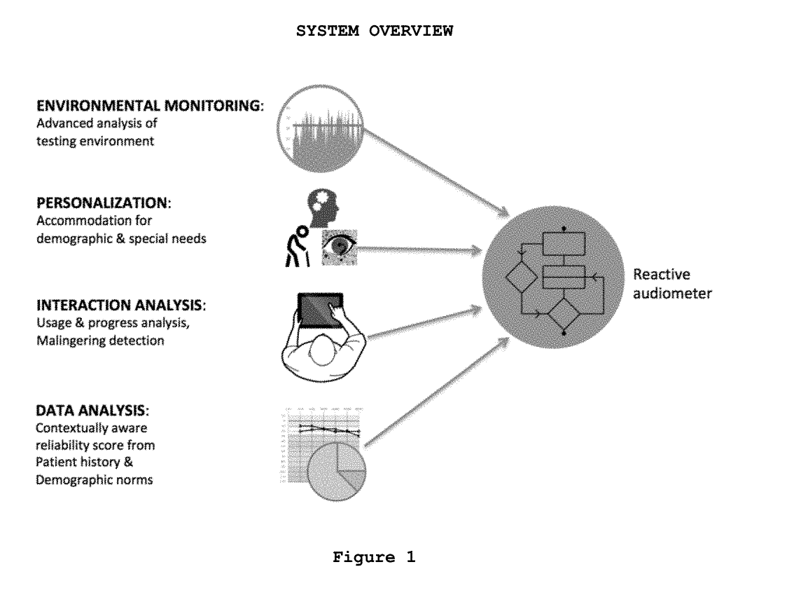

As displayed in FIG. 1, the present invention is capable of providing dynamic adjustments based on four (4) “input factors” that independently, or in some combination, comprise possible inputs for implementing a reactive audiometer, namely:
Environmental Monitoring
Patient Information/Preferences (Personalization)
Interaction Analysis
Data Analysis

Each of these four input factors may be individually referred to as an “input factor” or they may be collectively referred to in any combination as “input factors”.

The nature of each of the input factors is briefly described below as follows:
1. Environmental Monitoring.

Environmental monitoring in the present invention encompasses more than simply noise (decibel-level) monitoring. Whereas some newer audiometers known to persons skilled in the art are capable of monitoring and reporting on ambient background noise levels (some having the ability to either alert the operator or pause the hearing test if the background noise exceeds a certain defined level), environmental monitoring herein includes analysis of timing and constancy characteristics of background noise in relation to the state of the hearing test protocol, paying attention to critical and non-critical sections of the protocol in order to avoid unnecessarily pausing the test workflow. Environmental monitoring may also therefore include detection of distracting sound (not necessarily within the typically monitored frequency bandwidth) that may have a central masking affect (thus potentially affecting hearing thresholds). Other environmental monitoring may include measuring and analyzing factors such as temperature, humidity, lighting levels, and non-auditory vibrations to determine whether they (or any variations thereof) may be impacting the hearing test. Location information may also be sensed (via GPS or other means) and associated with other environmental readings or external data to determine or remember if a particular location is suitable for hearing testing. Minimally, these measurements can be saved for later statistical analysis or machine learning, for example, in order to construct variations of a reactive audiometer that suggests when the environment is suitable for conducting a hearing test, or to proactively detect adverse environmental conditions and adjust the audiometer's operation accordingly.

It is not necessary for the disclosure of the present invention to describe techniques for environmental monitoring, which would be within the ability of a person skilled in the art, but rather to describe a technique and method of analyzing and reacting to such environmental factors in order to dynamically adjust the operation of the portable audiometer during testing to be able to arrive at valid testing results.
2. Patient Information/Preferences (Personalization).

Disparate patient groups have different needs when testing, and human audiologists try to accommodate these needs. The present invention includes techniques for implementing a dynamically automated audiometer that is capable of modifying its operation based on knowledge of the patient's demographics, previously existing conditions (e.g. tinnitus, colour-blindness, mentation level, physical dexterity, existing hearing loss), prior noise exposure, usage of hearing-aids or assistive devices, general technology experience level, personal test protocol preferences, spoken or written languages, literacy level, cultural background, job description, prior hearing test performance and/or other such personalization factors.

It is not necessary for the disclosure of the present invention to describe techniques for receiving input of all possible patient information, which would be within the ability of a person skilled in the art, but rather to describe a technique and method of analyzing and reacting to such factors in order to dynamically adjust the operation of the portable audiometer during testing to be able to arrive at valid testing results.
3. Interaction Analysis.

By monitoring patient interactions with an automated audiometer, it is possible to automatically detect signs of patterned answers, malingering, inconsistent responses, misunderstanding of the task, attention level, and many other signs that a human audiologist looks for.

Interaction analysis could report on the pace and style (e.g. a telling way that a patient drags objects on the screen) of responses to distinguish between a patient who is getting bored of the test versus a patient who is having to take a long time responding due to hearing loss or physical limitations.

The disclosure of the present invention provides some examples of how an audiometer can detect these “signs”, and how to implement a reactive audiometer that is capable of dynamically adjusting its operation based on these detected factors. Dynamic adjustments may include, but are not limited to, providing timely assistance, redoing questionable thresholds, or simply annotating the test results with observations noted during the test and passing on this knowledge as inputs to other factors such as data analysis (described below).

It is not necessary for the disclosure of the present invention to describe all interaction analysis techniques, which would be within the ability of a person skilled in the art, but rather to describe a technique and method of analyzing and reacting to such interaction behaviour in order to, for instance, dynamically adjust the operation of the portable audiometer during testing to be able to arrive at valid testing results.
4. Data Analysis.

Even with best efforts to ensure that data is collected reliably during a hearing test, a post-test data analysis can be invaluable.

This can include analysis of presently collected test results (e.g. hearing test results, questionnaire responses, etc.) potentially in combination with analysis of previously acquired data (e.g. prior test results, clinical or demographic norms, etc.).

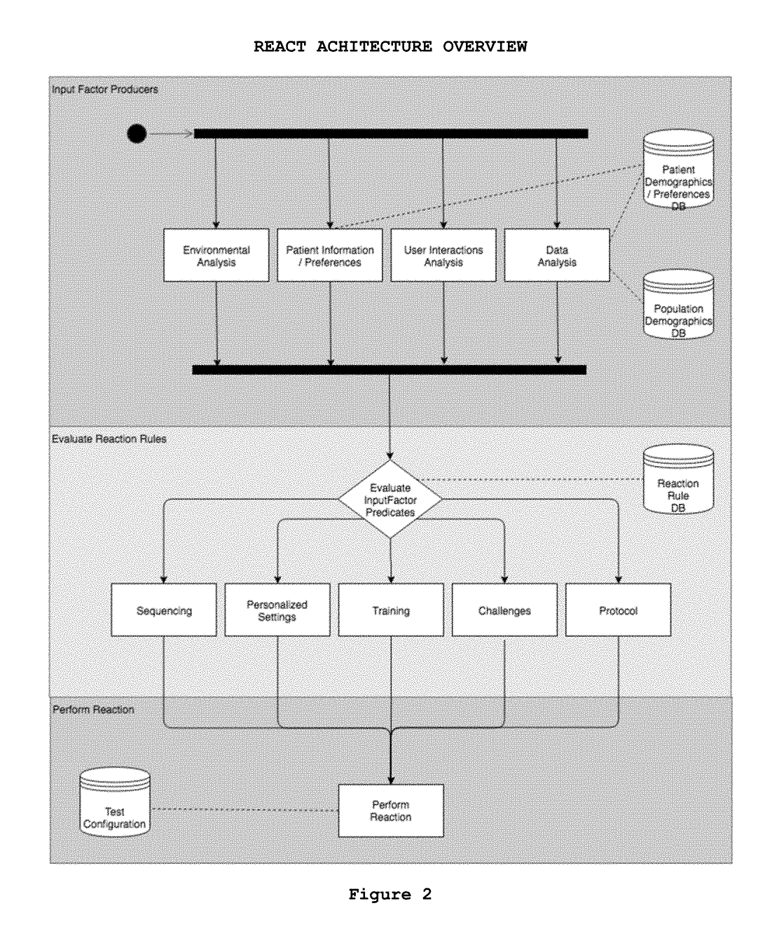

Data Analysis output can then be used by a reactive audiometer that is capable of dynamically adjusting its operation based on this analysis. One possible output of Data Analysis could be a reliability score or collection of scores, however insights from analysis could take any desired form that can be consumed by a Rule Processor (as shown in FIG. 2).

The present invention therefore includes a technique to use outputs of Data Analysis (such as reliability scores) to implement a reactive audiometer that can adjust its operation during the current or future tests to increase the probability of obtaining valid and accurate test results.

It is not necessary for the disclosure of the present invention to describe all possible data analysis techniques, which would be within the ability of a person skilled in the art, but rather to describe a technique and method of reacting to such analyzed data in order to be able to dynamically adjust the operation of the portable audiometer during future testing with the goal of increasing the chance of arriving at valid testing results.

### Reaction Types

To adjust the audiometer's operation in a manner that approximates or even improves upon adjustments that would be made by a human audiologist, the reactive audiometer of the present invention is capable of reacting to the above-mentioned input factors by adjusting its operation in accordance with five (5) “reaction types”, namely:
Sequencing
Personalized Settings
Training
Challenges
Protocol

The nature of each of the reaction types is briefly described below as follows:
1. Sequencing.

When input factors or manual intervention indicate that the test sequence needs to be modified, the Sequencing Reaction may be triggered. This may be due to ambient noise conditions, patient inputs, or other factors. In the case of a threshold-seeking algorithm, this could trigger the algorithm to repeat an individual presentation, redo a single approach to a threshold, redo an entire frequency, skip a frequency, add a new frequency, retest the entire audiogram, pause the test until an input factor changes (example: ambient noise), etc. For other test types like speech testing, it could control repeated presentation of words, or adjust the length of the test for example. Whatever the test type, the Sequencing Reaction controls how the current test proceeds.
2. Personalized Settings.

A human audiologist will sometimes tailor a test to a patient, especially if they have special needs, but they may also modify the test for a particularly capable patient. Some examples of personalization in a reactive audiometer are modified palettes suitable for the colour blind, personalized starting volume levels for pure-tone or speech tests when an existing hearing loss is already known, adaptive presentation speed based on patient reaction time, different interface modalities based on dexterity or intellectual capability of the patient, age-appropriate encouragement throughout the test, or any other modification to the test to accommodate the needs or optimize the experience for the patient.
3. Training.

A human audiologist will naturally detect when a patient requires additional instruction and can decide on the best way to present this instruction given the demographics of the patient.

When the reactive audiometer detects (as directed by afore-mentioned input factors) that additional training seems to be required, it has two general approaches: the first possibility may be to pause the test and simply notify the examiner (either locally or remotely) that additional training seems to be required; the second possibility may be to automatically present training materials directly to the patient. This is predicated by training materials being available to the system and the patient being of a suitable audience type to be capable of consuming the materials.

It should be noted, that the Reactive Audiometer may choose to immediately follow the Training reaction with a Challenge reaction (i.e. a quiz or practice task) to reinforce the training. In the case where the Challenges continue to be failed, the Protocol reaction may be invoked to switch to a more suitable test type (e.g. perhaps a simpler test or even an audiologist-assisted mode).
4. Challenges.

Challenges allow the reactive audiometer to confirm hypotheses. For example, if an input factor has detected that the user is responding via patterns, a challenge can be issued that is specifically designed to confirm or refute the suspicion. When a Modified Hughson Westlake algorithm is running, for example, this can take the form of presenting a tone outside of the normal sequence where the expected response is known, such as presenting at a volume level previously known to be easily detectable by the patient. Suspicions of malingering, task confusion, inattention, apparent dexterity, tinnitus, and other suspicions can be confirmed with challenges designed for each case. Challenges can also be used even before hearing tests begin to automatically generate personalization information such as a confirmation of dexterity levels, cognitive decline, etc. Challenges can also simply take the form of questionnaires that directly acquire patient information such as existing conditions, hearing history, personal preferences, etc.

These challenges are non-diagnostic in terms of hearing, and are targeted at learning about the patient's capability or willingness to perform the hearing test.

Practice tests or quizzes can also be triggered, perhaps after training has been completed to reinforce what was learned. This confirms to the audiometer that the patient understands the task and is capable and willing to perform it.
5. Protocol.

Beyond controlling aspects of a particular test, the human audiologist will also select a protocol of tests that are suitable to the patient's age, history, and test performance. While a default sequence is usually attempted initially, it will be modified as needed for the patient. For example, masking will only be applied during a pure-tone test when required by asymmetric hearing loss, and bone testing only when the air thresholds are high enough to warrant further investigation. Other tests like an audiologist-assisted Hughson Westlake test, or a Stenger test could be introduced when the reactive audiometer detects that malingering is occurring. Follow-on tests like SRT, Discrim, DTT, Speech-in-noise, psychoacoustic tests (ie. for CAPD) may be also recommended based on initial test results to further clarify or add more information to the results.

The reactive audiometer uses Data Analysis to trigger the Protocol reaction to recommend or automatically select appropriate follow-up test or questionnaires.

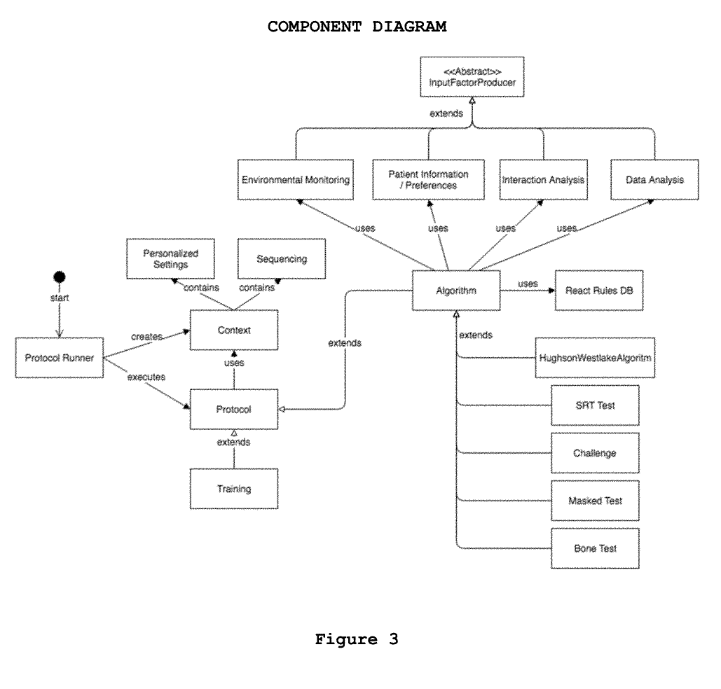

In view of the foregoing, and with reference to FIGS. 2 and 3, in one embodiment the present invention provides a reactive audiometer that is capable of dynamically adjusting an audiometer's operation by mapping Input Factors to Reactions via a Rule Processing block that can be implemented in more than one manner in order to dynamically control the operation of the audiometer to approach or exceed intelligent operation that could be achieved by a skilled audiologist. More specifically, in one embodiment the present invention provides a computer-implemented method for dynamically adjusting an audiometer's operation based on four (4) categories of input factors comprising steps of: (i) actively monitoring selected environmental factors before and/or during the test; (ii) freshly collecting or reloading pre-collected personalization preferences before the test; (iii) actively monitoring and analyzing the user's interactions with the audiometer during the test; (iv) scoring the reliability of the collected data; and (v) adjusting the operation of the audiometer based on analysis of one or more of the input factors.

The steps of actively monitoring environmental factors and adjusting the audiometer's operation based on this may comprise steps of: (i) using hardware-integrated or external microphones to determine the ambient noise levels at selected frequencies over selected time intervals; (ii) comparing the recorded measurements against sound levels and durations that are permitted to occur during the presently running portion of the hearing test, and/or comparing against previously recorded sound levels at this geographic location; (iii) determining if the measured ambient sound should prevent the test from proceeding to the next step, or if the current step should be invalidated; and (iv) pausing the test (until ambient noise levels improve, or until instructed by the user to attempt to continue), or immediately re-testing the invalidated step, or marking the step for subsequent retesting and immediately moving onto the next step of testing.

The steps of collecting or reloading personalization preferences and adjusting the audiometer's operation based on this may comprise steps of: (i) freshly collecting or reloading pre-collected personalization preferences before the test (including, but not limited to those preferences inferred from demographic information and/or existing medical conditions in addition to explicitly stated personal preferences); and (ii) personalizing the test in multiple ways including, but not limited to, adjusting the colour-palette for colour-blind patients, selecting pulsed or warble tone (rather than pure-tone) for children or patients with tinnitus, enabling interaction technique that require less precision for those patients with physical dexterity issues, selecting age-appropriate graphical themes and encouragements, selecting amongst various testing algorithm modes (such as self-paced or audiologist-assisted protocol) to accommodate age or mental agility needs, etc.

The steps of actively monitoring and analyzing the user's interactions and adjusting the audiometer's operation based on this may comprise steps of: (i) monitoring all patient-to-system interactions during the test including but not limited to detecting response patterns, malingering, inconsistent responses, misunderstanding of the task, attention drift, cognition issues, or detecting any other potentially negative patient behaviours that a human audiologist may watch for during a test; (ii) modifying the audiometer's operation to confirm the behaviour detection, including but not limited to presenting “challenges” to the patient designed to confirm or refute that the suspected patient behaviour is indeed occurring; (iii) modifying the audiometer's operation including, but not limited to, increasing the number of trials required to acquire a threshold to compensate for inconsistent responses or malingering; (iv) pausing the test and prompting for the attendant to intervene, or presenting instructional guidance to the patient, or presenting practice tests when misunderstanding or attention drift is detected; and (v) noting any unresolved observations and passing these observations onto the Data Analysis component as input factors, or adjusting the test protocol in response.

The steps of scoring the reliability of the collected data and adjusting the audiometer's operation based on this may comprise steps of: (i) collating presently collected data (such as air thresholds, bone thresholds, questionnaire responses, demographic information), environmental monitoring data, personalization settings, and interaction analysis data; (ii) collating previously collected data of the same type as in step (i); (iii) analysing the presently and previously collected data to produce a reliability score (or a collection of reliability scores each scoring different aspects of the data) using standard published techniques, custom rule sets, statistical analysis, machine learning, or other techniques; and (iv) modifying the audiometer's operation based on the calculated reliability score or scores including, but not limited to, recommending follow-up actions to the operator or patient, automatically retesting all or portions of the test, or annotating the results with the reliability score or scores.

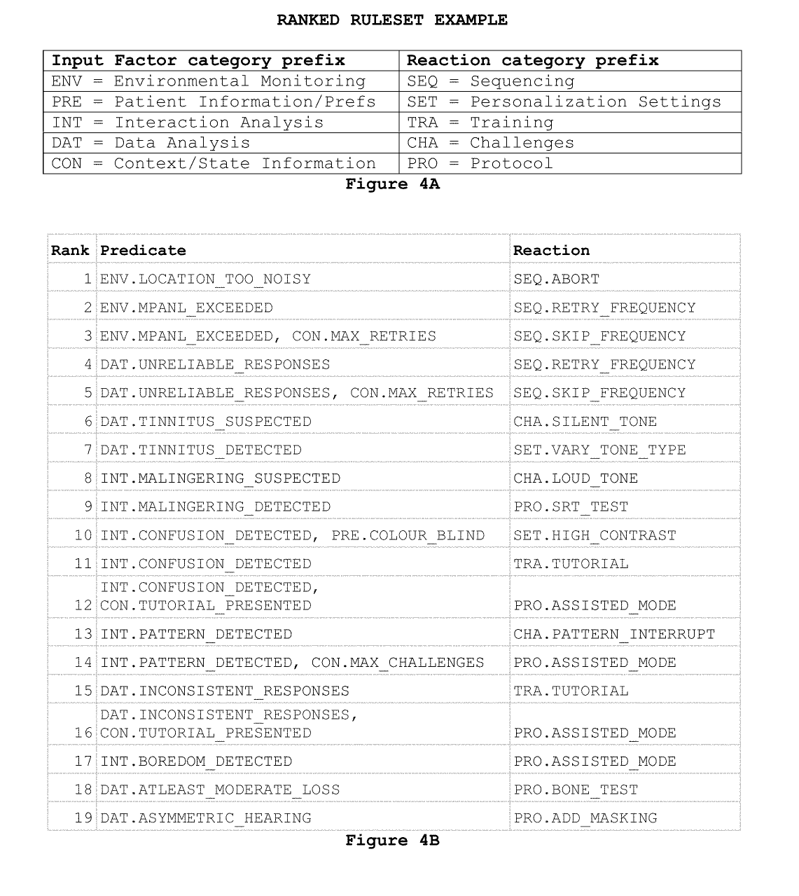

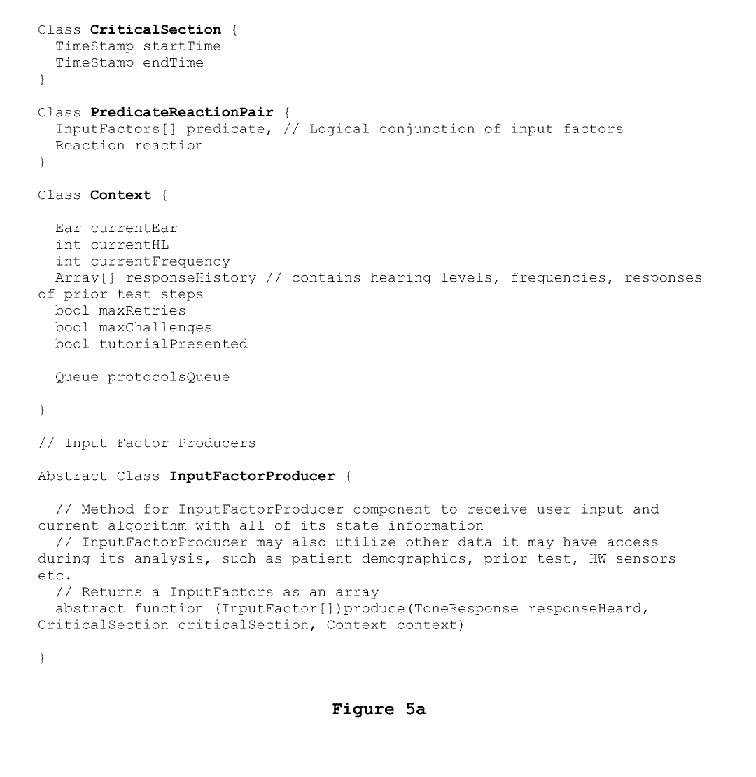

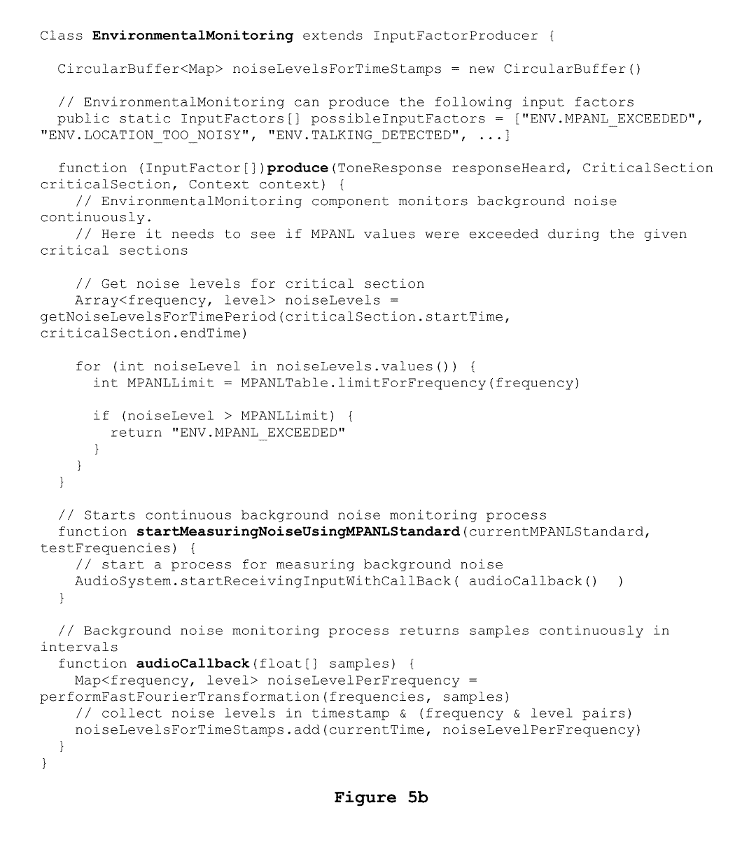

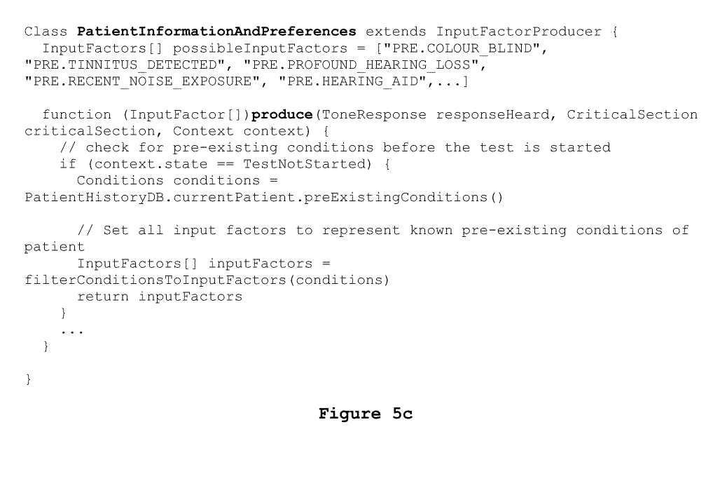

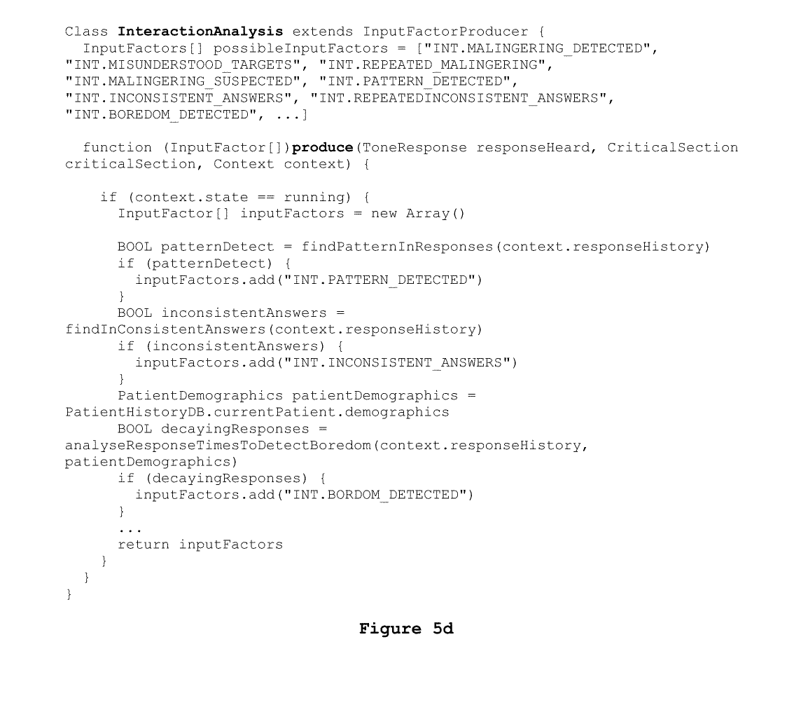

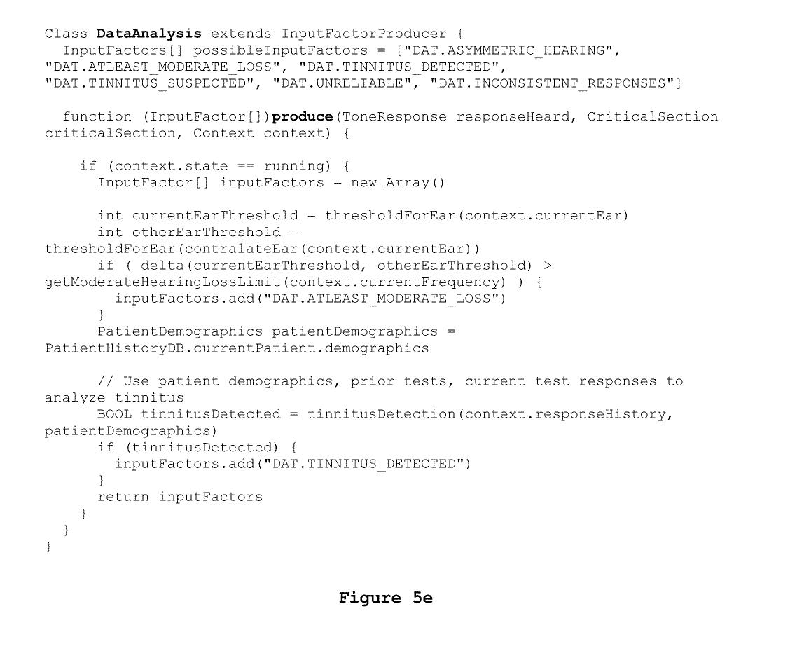

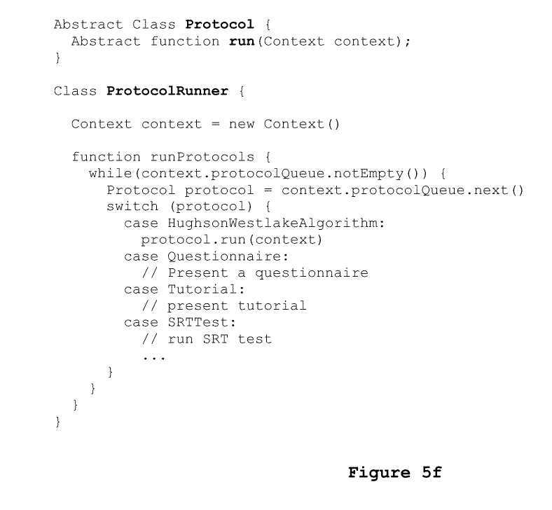

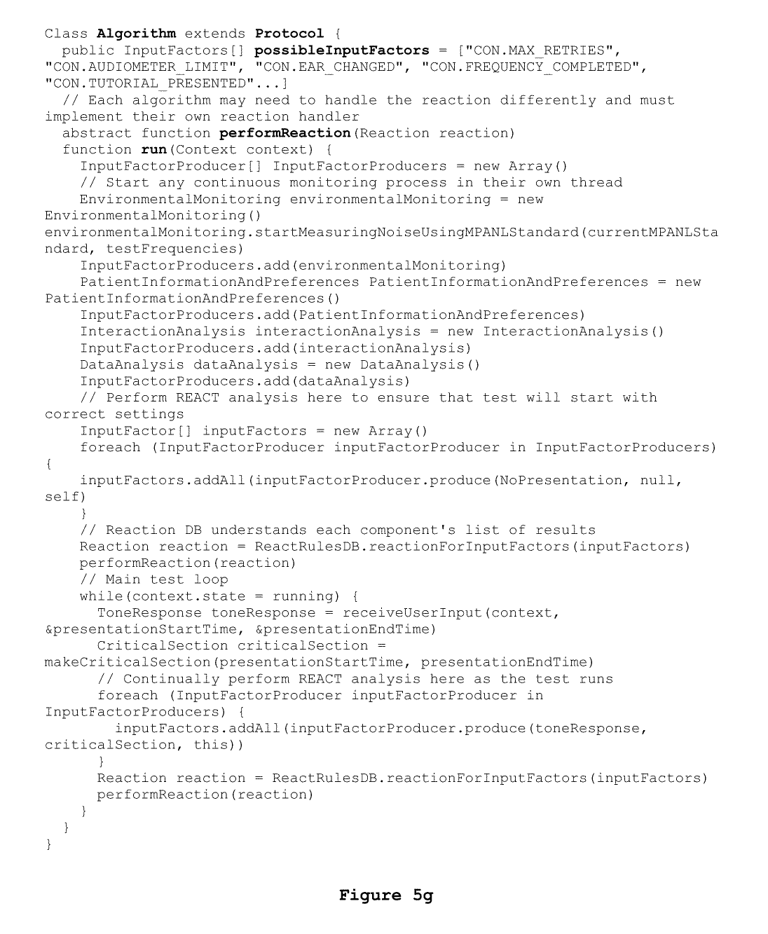

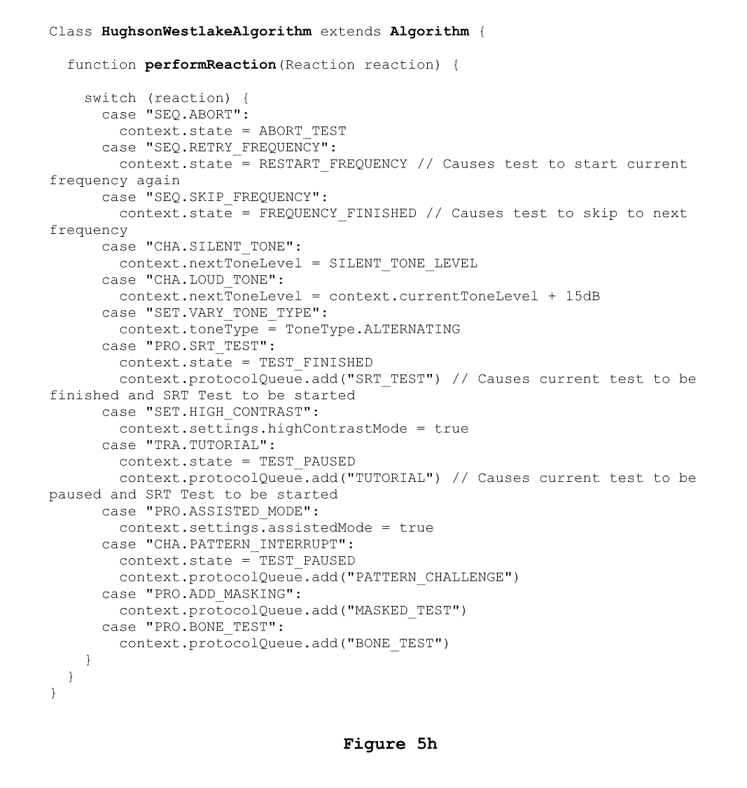

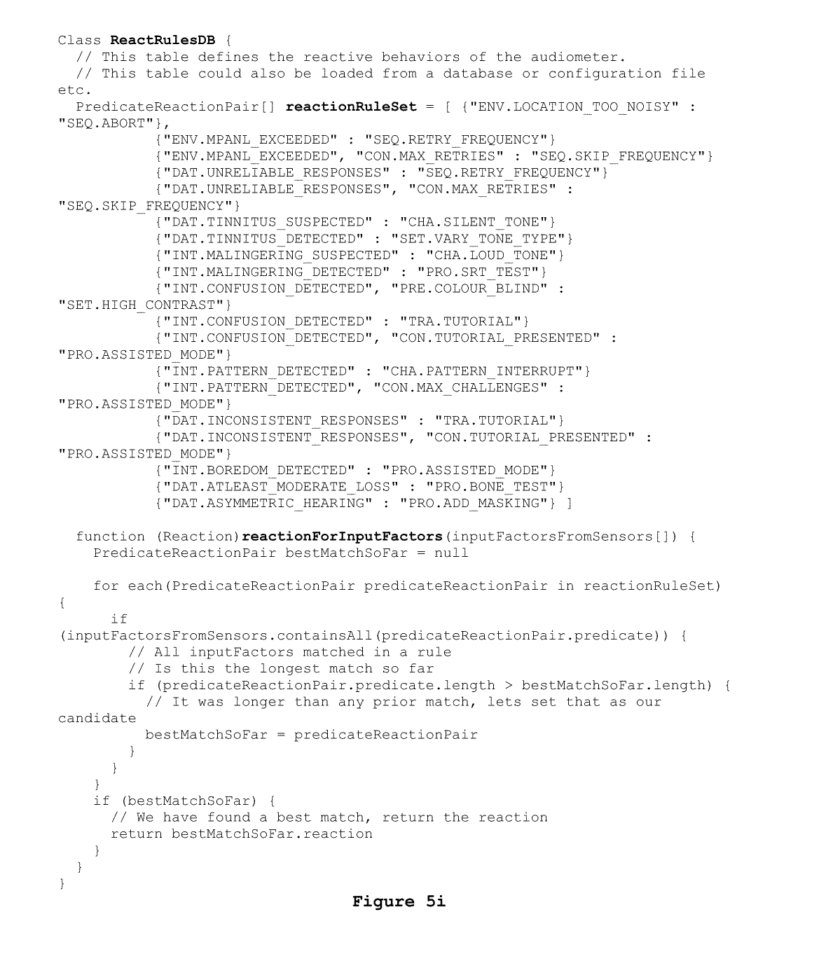

FIG. 4B displays an overview of an example Reactive Rule Set that may be implemented in accordance with the method of the present invention. For the purposes of demonstration, we have chosen to encode the rules as predicates containing input factors which are logically conjoined (ie. “AND” operator). In this example, the rules may be evaluated top-to-bottom looking for the first best-fit rule (predicate with the most matching terms) that evaluates to true; upon which the Reaction for that rule is run. This overall process, as generally depicted in FIG. 2, is continuously repeated while the audiometer is in operation. The Reactive Rules structure displayed in FIG. 4B could be populated in many ways, including by code (as shown in FIGS. 5a to 5i), or by loading from a database, loading a user-provided rule set, or generated dynamically from a machine learning system, for example. Other implementation techniques could use other non-table-based techniques for mapping inputs to outputs such as hardcoded logic statements run directly in code, neural net outputs, etc.

FIGS. 5a to 5i provide pseudocode examples that will allow a person skilled in the art to make and work the invention as a reactive rules structure by code.

FIG. 5a defines a CriticalSection class which is used to specify the time period within which ambient noise measurements are permitted to trigger a reaction. The PredicateReactionPair class is used as the building block of the reactionRuleSet table (see FIG. 5i). The Context class allows communication between the components and is used like a 5th “Input Factor” (referred to as “CON.x”) in the reactionRuleSet. The InputFactorProducer is an abstract class, that all input factors must implement, containing one abstract method called produce.

FIGS. 5b to 5e each show an implementation example of an input factor producer for each of the 4 categories of input factors. In FIG. 5b, the EnvironmentalMonitoring input factor producer can produce many possible input factors, which are tokenized representations of environmental situations such as excessive ambient noise, location previously known to be too noisy, distracting talking detected, etc. A few possible input factors that can be employed are listed in the possibleInputFactors structure. The other 2 functions in FIG. 5b are helpers for determining the ENV.MPANL EXCEEDED input factor. Additional methods would be implemented to support each additional input factor that is needed in the category of environmental monitoring.

In FIG. 5c, the PatientlnformationAndPreferences input factor producer can produce many possible input factors, which are simply tokenized representations of the patient's pre-existing conditions and preferences loaded using the preExistingConditions( )function from the patient's profile stored in a database.

In FIG. 5d, the InteractionAnalysis input factor producer is capable of producing a very large number of possible input factors. The result is essentially a group of synthetic sensors that encapsulate “best guess” descriptions of what the patient is doing. Multiple real sensor inputs can be used to build the input factors, and we show the steps for how to produce input factors for 3 of the example factors listed in the possibleInputFactors structure. In this respect, in FIG. 5d we show the pattern and timing of patient responses being used to find INT.PATTERN_DETECTED (meaning the patient isn't properly performing the hearing test), INT.INCONSISTENT_ANSWERS (by detecting larger-than-typical variability in the threshold after repeated attempts to determine it), and INT.BORDOM_DETECTED (by looking for patterns in response times such as large delays in responses, multiple unrealistically fast responses in a row, or any other patterns that are known by audiologists to indicate a lack of attention or playing around).

In FIG. 5e, we show how to generate a couple of example input factors from DataAnalysis. This example shows the steps for generating the input factors DAT.ATLEAST_MODERATE_LOSS (by simply comparing the hearing thresholds between the ears to see if they differ by some pre-configured amount) and DAT.TINNITUS_DETECTED (combines data from several sources using some tinnitusDetection( ) function (a simple implementation of which could just return TRUE for example if the patient answered “YES” to a questionnaire asking “Do you often hear ringing in your ears?”, and more intelligent versions could look for a history of inconsistent responses at a particular frequency) to draw the conclusion that tinnitus is present). A similar structure can be used to generate any other input factor that depends on data analysis such as DAT.UNLRELIABLE, DAT.INCONSISTENT_RESPONSES, etc.

FIG. 5f provides pseudocode for the general workflow of the present invention. The audiometer's main loop executes a series of queued protocols. The reactive audiometer is set into action by queuing the first protocol, for example a “Hughson Westlake puretone test”, into the context.protocolQueue. Note that FIG. 5h contains reactions that can also add to this protocol queue (such as context.protocolQueue.add(“BONE_TEST”)), and thus extend the duration of the main loop. In FIG. 5f, this main loop runs until the protocolQueue is empty, at which point the audiometer returns to its resting state and awaits instructions from the operator.

In FIG. 5g, pseudocode is provided respecting how an Algorithm abstract class may be defined. It should be noted that protocols need not be hearing test algorithms; for example, a questionnaire is a protocol but not an algorithm. In FIG. 5g we show how an Algorithm abstract class may be defined as an extension of the Protocol class. Each Algorithm needs to define its own performReaction implementation (hence the abstract performReaction definition) because reactions can be very specific to the type of algorithm. The run function provides an example implementation that initializes threads to run the EnvironmentalMonitoring, PatientlnformationAndPreferences, InteractionAnalysis, and DataAnalysis input factor producers. For each input factor producer, the input factors are added to the inputFactors array in preparation for passing them to the performReaction function that is shown in FIG. 5h. In FIG. 5g, the performReaction function is also run once before the test algorithm loop starts to ensure that PatientlnformationAndPreferences can have a chance to influence how the test is configured before it starts.

FIG. 5h provides pseudocode showing an example of how a HughsonWestlakeAlgorithm might define its performReaction function. Here, we provide an example of handling reactions from each of the categories shown in FIG. 4a, namely some that alter the Sequencing, some that adjust the Personalization Settings, some that trigger Training, some that trigger Challenges, and some that add more Protocols to the protocol queue.

FIG. 5i provides pseudocode showing a possible implementation that defines the rules shown in FIG. 4b into an array called reactionRuleSet. We also show an example implementation of how to choose which rule to apply given a list of input factors. In reactionForInputFactors, we treat the list of rules as a first-best-match list, so we choose the first rule with the most matching terms in the predicate. Many other methods of mapping input factors to reactions are possible, and would be known to persons skilled in the art.

Although specific embodiments of the invention have been described, it will be apparent to one skilled in the art that variations and modifications to the embodiments may be made within the scope of the following claims.

We claim:

1. A computer-implemented method for dynamically adjusting the operation of an automated audiometer during a hearing test of a patient comprising the steps of:
(i) collecting or loading pre-collected personalization settings of the patient before the hearing test;
(ii) adjusting parameters of the hearing test to accord with the personalization settings of the patient;
(iii) commencing the hearing test;
(iv) actively monitoring and analyzing at least one input factor during the hearing test;
(v) adjusting operation of the hearing test if the at least one input factor meets a pre-defined triggering scenario;
(vi) repeating steps (iv) to (v) until the hearing test has been completed by the patient or stopped by the audiometer; and
(vii) analyzing results of the hearing test.

2. The computer-implemented method of claim 1 wherein the personalization settings of the patient comprise any one or more of demographics, previously existing medical conditions, including tinnitus, colour-blindness, mentation level, physical dexterity, and existing hearing loss, prior noise exposure, usage of hearing-aids or assistive devices, general technology experience level, personal test protocol preferences, spoken or written languages, literacy level, cultural background, job description, and prior hearing test performance.

3. The computer-implemented method of claim 1 or 2 wherein the step of adjusting parameters of the hearing test to accord with the personalization settings of the patient comprises any one or more of adjusting a colour-palette when the patient is colour-blind, selecting pulsed or warble tone when the patient has tinnitus, enabling an interaction technique that requires less precision when the patient has physical dexterity limitations, selecting age-appropriate graphical themes and encouragements for the patient, and selecting testing algorithm modes, including self-paced or semi-assisted modes, to accommodate age or mental agility needs of the patient.

4. The computer-implemented method of claim 1 wherein the at least one input factor comprises one or more of an environmental factor or interactions of the patient with the audiometer.

5. The computer-implemented method of claim 1 wherein the at least one input factor comprises one or more of an environmental factor and interactions of the patient with the audiometer.

6. The computer-implemented method of claim 4 wherein the at least one input factor is one or more of an environmental factor.

7. The computer-implemented method of any one of claims 1 to 6 wherein the one or more environmental factor comprises any one or more of ambient background noise (decibel-level), timing and constancy characteristics of background noise, distracting sound, temperature, humidity, lighting levels, and non-auditory vibrations.

8. The computer-implemented method of claim 4 wherein the at least one input factor is one or more of interactions of the patient with the audiometer.

9. The computer-implemented method of any one of claims 1 to 5 and 8, wherein the one or more interactions of the patient with the audiometer comprises any one or more of patterned answers, malingering, inconsistent responses, misunderstanding of a task, attention level, and pace and style of responses.

10. The computer-implemented method of claim 5 or 6 wherein the steps of actively monitoring and analyzing the one or more environmental factor and adjusting operation of the hearing test comprises the steps of:
(i) using hardware-integrated or external microphones to ascertain and record ambient noise levels at selected frequencies over selected time intervals;
(ii) comparing the recorded ambient noise levels against sound levels and durations that are permitted to occur during the hearing test;
(iii) determining if the recorded noise levels should prevent the hearing test from proceeding to a next step, or if a current step should be invalidated; and
(iv) based on the determination at step (iii), either pausing the hearing test until ambient noise levels improve to an acceptable level, or immediately re-testing an invalidated step, or marking the current step for subsequent retesting and immediately moving on to the next step of testing.

11. The computer-implemented method of claim 5 or 8 wherein the steps of actively monitoring and analyzing the one or more interactions of the patient with the audiometer and adjusting operation of the hearing test comprises the steps of:
(i) monitoring patient interactions with the audiometer during the hearing test to detect any one or more of response patterns, malingering, inconsistent responses, misunderstanding of the task, attention drift, and cognition issues;
(ii) determining if the patient interactions meets the pre-defined triggering scenario for the one or more of response patterns, malingering, inconsistent responses, misunderstanding of the task, attention drift, and cognition issues; and
(iii) if the pre-defined triggering scenario for the one or more of response patterns, malingering, inconsistent responses, misunderstanding of the task, attention drift, and cognition issues is met at step (ii), modifying operation of the audiometer in accordance with pre-defined responses to the triggering of the one or more of response patterns, malingering, inconsistent responses, misunderstanding of the task, attention drift, and cognition issues.

12. The computer-implemented method of claim 11 wherein the step of modifying operation of the audiometer in accordance with pre-defined responses comprises presenting challenges to the patient designed to confirm or refute presence of one or more of response patterns, malingering, inconsistent responses, misunderstanding of the task, attention drift, and cognition issues.

13. The computer-implemented method of claim 12 wherein operation of the hearing test is further modified to increase a number of hearing trials required to acquire a threshold to compensate for malingering or inconsistent responses, when malingering or inconsistent responses has been confirmed.

14. The computer-implemented method of claim 11 wherein the step of modifying operation of the audiometer in accordance with pre-defined responses comprises any one of pausing the test and prompting for an attendant to intervene, presenting instructional guidance to the patient, or presenting practice tests if misunderstanding of the task or attention drift is detected.

15. A computer program product comprising a computer readable memory storing computer executable instructions thereon that when executed by a computer performs the method steps of any one of claims 1 to 14.

16. A portable computer that performs the computer-implemented method of any one of claims 1 to 14.

17. A computer-based audiometer that performs the computer-implemented method of any one of claims 1 to 14.

18. A computer-implemented method for dynamically adjusting the operation of an automated audiometer during a hearing test of a patient comprising the steps of:
(i) defining input factor producers from one nr more categories of Environmental Monitoring, Patient Information, Interaction Analysis, and Data Analysis, whereby each such input factor producer is capable of continuously monitoring a defined set of sensor or data inputs for a pre-defined triggering scenario during operation of the hearing test;
(ii) defining a reaction that may occur during operation of the hearing test upon triggering of the pre-defined triggering scenario for each such input factor producer;
(iii) encoding the pre-defined triggering scenario and corresponding reaction for each input factor producer into a ruleset that defines which reaction or sequence of reactions should occur for each pre-defined triggering scenario;
(iv) commencing the hearing test and running the input factor producers;
(v) continuously evaluating the input factor producers, whereby if a pre-defined triggering scenario for an input factor producer is met, an input factor token for the triggered input factor producer is placed into an input factor container;
(vi) continuously evaluating the ruleset based on contents of the input factor container to determine which reaction or reactions should be executed, executing those pre-defined reactions indicated by the ruleset, and clearng the input factor container; and
(vii) repeating steps (v) to (vi) until the hearing test is ended.
